(19) United States
(12) Patent Application Publication
(10) Pub. No.: US 2025/0367106 A1
(43) Pub. Date: Dec. 4, 2025

(54) Topical Formulation for Improving Skin Quality and Inducing the Release of Oxytocin

(71) Applicant: OX Technology IP LLC, Chicago, IL (US)

(72) Inventors: Steven H. Dayan, Chicago, IL (US); Sabrina Fabi, San Diego, CA (US)

(21) Appl. No.: 18/733,782

(22) Filed: Jun. 4, 2024

(51) Int. Cl. A61K 8/9789; A61K 8/55; A61K 8/64; A61Q 19/08

(52) U.S. Cl. CPC A61K 8/9789; A61K 8/553; A61K 8/64; A61Q 19/08

(57) ABSTRACT

A formulation that creates a hormonal response in the body through a topical application. The topical formulation contains botanicals in the form of jasmine flower extract and carob fruit extract. The botanicals are mixed with phospholipids and peptides to promote the absorption of the botanicals into the skin. Once applied to the skin, the jasmine flower extract and the carob fruit extract induce a hormonal response in the body. The hormonal response stimulates the oxytocin neural pathways in the body. This causes the body to believe that oxytocin is present, therein triggering the release of oxytocin within the body. The presence of the oxytocin facilitates physical and pro-social psychological improvements that include an improved libido, improved self-esteem, and improved self-confidence.

### BACKGROUND OF THE INVENTION

### 1. Field Of The Invention

In general, the present invention relates to topical compounds that are used to improve the quality and appearance of skin. The present invention also relates to topically applied compounds that induce a hormone reaction, therein improving secondary elements, such as perceived attractiveness and improved sexual health.

### 2. Prior Art Description

As a person ages, it is not unusual for the skin to lose elasticity, become dry and develop areas of uneven pigmentation. To address these problems, numerous creams, serums, and other topically applied products have been developed, in both the pharmaceutical and over-the-counter markets. Skin aging is an ongoing process that varies widely between people with different skin types. Furthermore, skin aging is influenced by a complex interplay of environmental variables such as sun exposure, nutrition, diet, medications, and tobacco use.

Commercially available products often combine various epidermal and dermal regenerative stimulators with a direct aim to improve skin quality. This can produce not only physical benefits, but also psychological benefits that are associated with perceive improvements in appearance. That is, the better a person believes they appear, the more self-confidence and self-esteem that person has. Such positive patient mindsets are desired with any skin care treatment and skin care formulations are designed to create such positive patient mindsets indirectly. Since improved skin appearance is closely linked to higher self-esteem and enhanced quality of life, skincare products are often formulated with ingredients such as floral scents, that are intended to reinforce the positive psychological benefits associated with product use but do not directly stimulate any positive emotional triggers.

Oxytocin is a natural hormone that manages key aspects of the reproductive system, as well as aspects of human behavior. In the body, the hypothalamus makes oxytocin. The posterior pituitary gland stores and releases the oxytocin into the bloodstream. Oxytocin acts as a chemical messenger in the brain and has an important role in many human behaviors and social interactions, including sexual arousal, recognition, trust, romantic attachment, and parent-infant bonding. Oxytocin is one of a few hormones that have a positive feedback loop. This means that the release of oxytocin leads to actions that stimulate your pituitary gland to release even more oxytocin over time. Thus, if the release of even a small initial amount of oxytocin can be induced, the effects can multiply and have significant effects on a person.

In the prior art, an oxytocin reaction is induced by administering synthetically manufactured oxytocin to a person either intravenously or through nasal inhalation. See U.S. Patent No. 11,291, 26, to Djupesland. In such uses, oxytocin is a controlled pharmaceutical. As such, administration of oxytocin requires medical need and a prescription or administration by a medical professional.

The present invention is a topical formulation that utilizes ingredients that are considered GRAS “generally recognized as safe” by the United States Food and Drug Administration (FDA). As such, the formulation can be made and sold on the open market without need of prescription. The topical formulation contains ingredients derived from jasmine in particular that stimulate the oxytocin neural pathways in the body. The jasmine extract causes an increase in oxytocin levels in the skin as well as signals via mechanosensory neuronal cells to the body that oxytocin is present, therein triggering the release of oxytocin within the body. The result is a topical formulation that not only conditions the skin but also provides the positive psychological effects associated with oxytocin in the body. The details of the present invention are described and claimed below.

### SUMMARY OF THE INVENTION

The present invention is a formulation and the associated method of creating a hormonal response in the body through the topical application of the formulation. The topical formulation contains botanicals in the form of jasmine flower extract and carob fruit extract. The botanicals are mixed with phospholipids and peptides to promote the absorption of the botanicals into the skin.

Once applied to the skin, the jasmine flower extract and the carob fruit extract induce a hormonal response. The hormonal response stimulates the same neural pathways in the body, as does oxytocin. The stimulation of the neural pathways in addition to the stimulated mechano-sensory receptors causes the body to believe that oxytocin is present. This triggers the release of oxytocin and/or similar hormones within the body. The presence of the hormones facilitates physical and psychological improvements that include an improved libido, improved self-esteem, and improved self-confidence.

The botanicals are also mixed with a formulation that improves the health and appearance of the skin. As such, self-esteem and self-confidence are further increased by an overall improvement in the treated skin.

### BRIEF DESCRIPTION OF THE DRAWINGS

For a better understanding of the present invention, reference is made to the following description of an exemplary embodiment thereof, considered in conjunction with the accompanying drawings, in which:

FIG. 1 shows an ingredient list along with percentage by weight used to create the present invention topical formulation;

FIG. 2 shows a series of histograms that show percent increases in various physical and psychological factors after use of the present invention topical formulation;

FIG. 3 is a pair of histograms that show percent increases in perceived attractiveness and trustworthiness in others caused by the use of the present invention topical formulation;

FIG. 4 shows a histogram illustrating a percent increase in perceived attractiveness of others after use of the present invention topical formulation; and

FIG. 5 shows a list of survey categories and a histogram showing improvements in survey categories attributed to use of the present invention topical formulation.

### DETAILED DESCRIPTION OF THE DRAWINGS

Although the present invention topical formulation can be embodied in many ways, only one exemplary embodiment is illustrated. The exemplary embodiment is being shown for the purposes of explanation and description. The exemplary embodiment is selected in order to set forth one of the best modes contemplated for the invention. The illustrated embodiment, however, is merely exemplary and should not be considered a limitation when interpreting the scope of the appended claims.

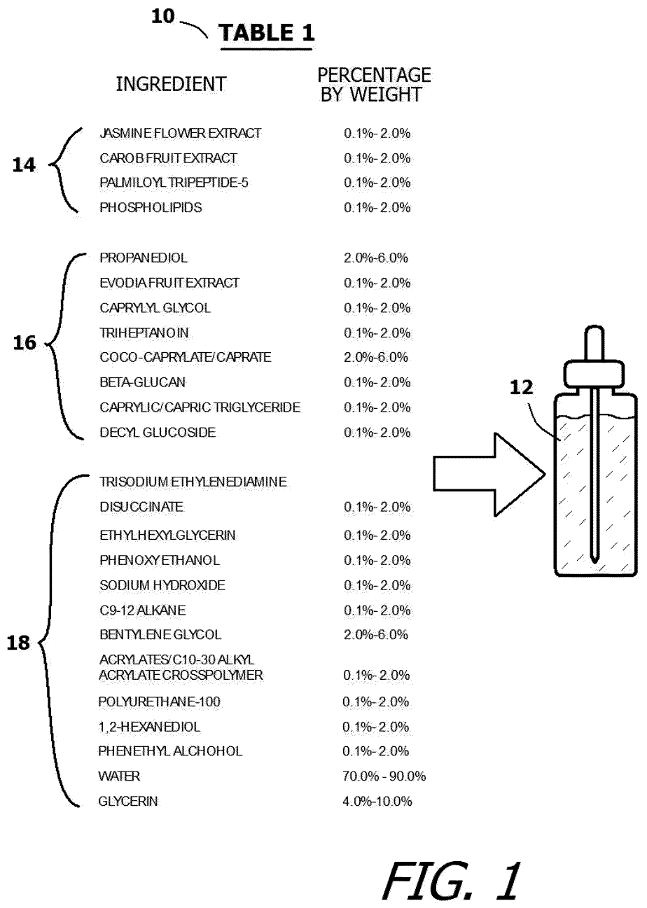

Referring to FIG. 1, an ingredient list 10 is provided in Table 1 that is used to produce a topical formulation 12. The topical formulation 12 is designed to be applied to the skin as a serum or liquid. The topical formulation 12 can also be incorporated into secondary products, such as cosmetics. It will be understood that the formulation presented in FIG. 1 is for a serum or liquid. If the present invention were embodied as a cream, moisturizer, lip balm or the like, the formulation would be varied. However, the primary active ingredients would remain the same.

The exemplary ingredient list 10 contains a first set 14 of active ingredients that embody psycho-social-dermatological benefits. The first set 14 of ingredients work in synergy to stimulate the release of oxytocin and/or like hormones in the body. The ingredient list 10 also contains a second set 16 of active ingredients that provide purely dermatological benefits. That is, the second set 16 of ingredients firm, moisturize and soften the skin. Lastly, the ingredient list 10 contains a third set 18 of ingredients that are mostly inert and are used to dissolve, preserve, and control the viscosity of the active ingredients.

The first set 14 of active ingredients include jasmine flower extract from the species Jasminum Officinale. The jasmone flower extract has primary and secondary effects. The secondary effects include a pleasant scent and scents the overall topical formulation 12. Furthermore, the secondary effects include the fact that jasmine flower extract serves as both an antioxidant and an anti-inflammatory agent to the skin. The more important primary effect is that the jasmine flower extract effects the hormonal and pheromonic pathways when absorbed into the skin. This stimulates the body into believing that certain hormones and/or pheromones are present. If the hormone and/or pheromone has a reactive feedback loop, such as oxytocin, then the belief that the hormone/pheromone is present will cause an actual release of the hormone/pheromone. This can produce an increase in hormone and/or pheromone levels in the skin. This effect can be increased by combining the jasmine flower extract with the other active ingredients of carob fruit extract and phospholipids.

The carob fruit extract comes from the fruit of a carob tree. The carob tree is an evergreen tree belonging to the legume family and is common to Western Asia and the Mediterranean basin. Carob fruit extract contains isobutyric acid and inositol. In addition, the carob fruit extract includes a tanning stimulant and antioxidant. The isobutyric acid is a fatty acid similar to human vaginal secretions known as copulins. Copulins act as an important messenger or pheromone in primates signaling reproductive receptivity. Both the odorous and non-odorous chemical compositions within copulins can trigger behavioral and physiological responses in males, wherein males exposed to copulins perceive themselves as more sexually desirable.

In addition, the inositol present in the carob fruit extract protects the skin from photoaging. The inositol activates the synthesis of collagen in the fibroblasts. Collagen is a fibrous protein present in the dermis that ensures the skin's flexibility and firmness. Inositol reinforces the stock of collagen and thus compensates for part of its degradation during exposure to the sun. Furthermore, inositol intensifies and prolongs the skin's tan, based on the activation of secondary messengers involved in melanin synthesis. On a biological level, it regulates the Diacylglycerol/Protein Kinase C (DAG/PKC) pathway. Diacylglycerol (DAG) is a secondary messenger, signal transducer, synthesized from membrane phosphoinositols. Inositol activates protein kinase C (PKC) in a number of tissues. The PKC system activates tyrosine, the key enzyme in melanin synthesis, by phosphorylating its cytoplasmic domain, particularly on serine residues. Thus, inositol stimulates and prolongs tanning.

The jasmine flower extract and the carob fruit extract both produce hormonal/pheromonal responses in the body. The effectiveness of the jasmone flower extract and the carob fruit extract are enhanced by being combined with phospholipids. Phospholipids are types of lipids composed of glycerol, fatty acids, and phosphate. Phospholipids are hygroscopic, meaning phospholipids absorb water from surrounding air and hold it where more hydration is needed. Applying phospholipids to skin can help replace phospholipids that are depleted either through cleansing or from environmental factors. The phospholipids assist the skin in absorbing the jasmine flower extract and the carob fruit extract, therein making the hormonal responses to the jasmine flower extract and the carob fruit extract more effective.

The combination of phospholipids with the jasmine flower extract and the carob fruit extract work synergistically to both improve skin quality, and to trigger a hormonal response. The release of hormones that include oxytocin produces an improved sense of self-confidence and self-esteem. The combined jasmine flower extract, carbo fruit extract and phospholipids react through the Piezo-1 receptors in the skin. The skin transmits the sensation of touch to sensory neurons through the Piezo-1 receptors found in the plasma membrane of epidermal keratinocytes and fibroblasts. In addition to triggering increased oxytocin levels, such receptor stimulation is integral to the skin's wound healing process, modulating inflammation, tissue proliferation, and scar formation. Jasmine flower extract, in particular, influences Piezo-1 receptors when applied topically.

Peptides can also be mixed with the jasmine flow extract, carob fruit extract, and phospholipids. The peptides further increase the ability of the skin to absorb the active ingredients. The listed embodiment utilizes Palmitoyl Tripeptide-5 as an exemplary peptide. Palmitoyl Tripeptide-5 is a synthetic, signal peptide made from the amino acids lysine and valine combined with the fatty acid palmitic acid, which aids penetration. It has a visible impact on skin's firmness and also helps smooth the appearance of fine lines and wrinkles, including deep wrinkles. Palmitoyl Tripeptide-5 supports the skin's extracellular matrix, including collagen and hyaluronic acid, so skin looks smoother and more youthful. Palmitoyl Tripeptide-5 can also help inhibit environmentally-generated damaging enzymes in skin that degrade its firmness and resilience.

The second set 16 of ingredients are primarily directed toward effecting the condition of the skin. The second set 16 of ingredients includes propanediol, also known as 1, 3-propanediol. Propanediol is a humectant that draws moisture into the skin.

Propanediol is a glycol and offers moisturizing properties that can result in a smooth, dewy finish as the topical formulation 12 dries on the skin. The second set 16 of ingredients also uses Caprylyl glycol as a humectant in addition to the Propanediol. Caprylyl glycol is a skin conditioning agent that can either be plant-derived or synthetic.

Triheptanoin is used in the topical formulation 12 as a skin-softening emollient. Triheptanoin is made from a blend of glycerin and heptanoic acid. The lightweight emollient feel of triheptanoin makes it a good stand-in for siloxane-type silicones such as clopentasiloxane, which can be used in the alternative. Triheptanoin smooths and softens the skin as it prevents moisture loss. Triheptanoin is also used as a dispersing agent for other ingredients, ensuring that the other ingredients are distributed evenly. In addition, to the triheptanoin, coco caprylate/caprate is also utilized as a lightweight emollient for softening the skin.

The topical formulation 12 preferably includes beta-glucan. Beta-glucan is a polysaccharide that can be derived from various natural sources such as yeast, fungi, seaweed, or oats. Beta-glucan is a soothing agent and minimizes redness and other signs of sensitive skin. Beta-glucan also offers antioxidant properties and has been shown to help visibly minimize signs of aging/sun damage including reducing the look of wrinkle depth. Certain sources of beta-glucan also offer film-forming moisturizing properties.

Evodia fruit extract is included in the topical formulation 12. The Evodia fruit extract is an anti-inflammatory agent that inhibits the skin from reddening of otherwise adversely reaction to the other ingredients.

Caprylic triglyceride is included in the topical formulation 12. Caprylic triglyceride is a compound that comes from combining fatty acids with glycerin. The fatty acids in the compound are medium-chain triglycerides that come from rich oils, such as coconut or palm oil. The caprylic triglyceride creates a barrier on the skin that traps moisture within the skin, therein moisturizing and softening the skin.

Decyl glucoside is used in the topical formulation 12 as a surfactant. Decyl glucoside attracts impurities and removes them from the skin surface when used with water. Decyl glucoside is preferred because of its property of deep penetration. In the skin, decyl glucoside decreases fine lines and wrinkles by hydrating through all the layers of skin. Decyl glucoside keeps feeding the skin with hydration which diminishes the appearance of fine lines. Furthermore, decyl glucoside is an effective emulsion stabilizer that enables the water and oils to mix.

The third set 18 of ingredients are considered inert and are utilized to control the viscosity and feel of the topical formulation 12. The topical formulation 12 is an aqueous solution. As such, the primary ingredient is water. The water is mixed with glycerin to bring the solution to a desired viscosity. Accordingly, water and glycerin are the most prevalent ingredients and act as carriers for the active ingredients.

Trisodium ethylenediamine disuccinate is added to the topical formulation 12 as a chelating agent. The trisodium ethylenediamine disuccinate binds with metal ions to prevent the metal ions from adhering to the surface of the skin or causing contamination.

Ethylhexylglycerin is used in the topical formulation 12 as a synthetic skin-softening agent. Ethylhexylglycerin is also a preservative and carrier/suspending agent that boosts the efficacy of other preservatives such as phenoxyethanol. It has been shown to have mild humectant properties leading to improved skin hydration without a sticky feel.

Sodium hydroxide is used in the topical formulation 12 to control pH. Sodium hydroxide, also known as lye, is highly alkaline that is used in small amounts in cosmetics to establish and hold the pH of a product in an acidic or alkaline formula.

C9-12 Alkane is provided as a solvent for dissolving other ingredients in a topical formulation 12 such that the ingredients mix well to form a rich and smooth texture. C9-12 Alkane also enhances the spreadability of the product and prevents the oil-based and water-based ingredients from separating. Butylene glycol is also used as a solvent that can soften and moisturize the skin. Butylene glycol acts as a solvent for ingredients and enhances their stability, texture, and absorption.

Acrylates/C10-30 alkyl acrylate crosspolymer is added to the topical formulation 12 primarily to control texture. Acrylates/C10-30 alkyl acrylate crosspolymer is a synthetic ingredient used to adjust texture and the perceived feel of the topical formulation 12.

Polyurethane-100 is added to the topical formulation to enable the topical formulation 12 to produce a continuous film on skin.

The ingredient 1,2-Hexanediol is utilized as a synthetic preservative, solvent, and moisture-binding agent. Phenethyl Alcohol is also used as a preservative. The 1,2-Hexanediol and phenethyl alcohol help extend shelf life and protects against the growth of harmful microorganisms. Additionally, apart from acting as a preservative, phenethyl alcohol adds a floral scent to the topical formulation 12 that complements that of the jasmine flower extract.

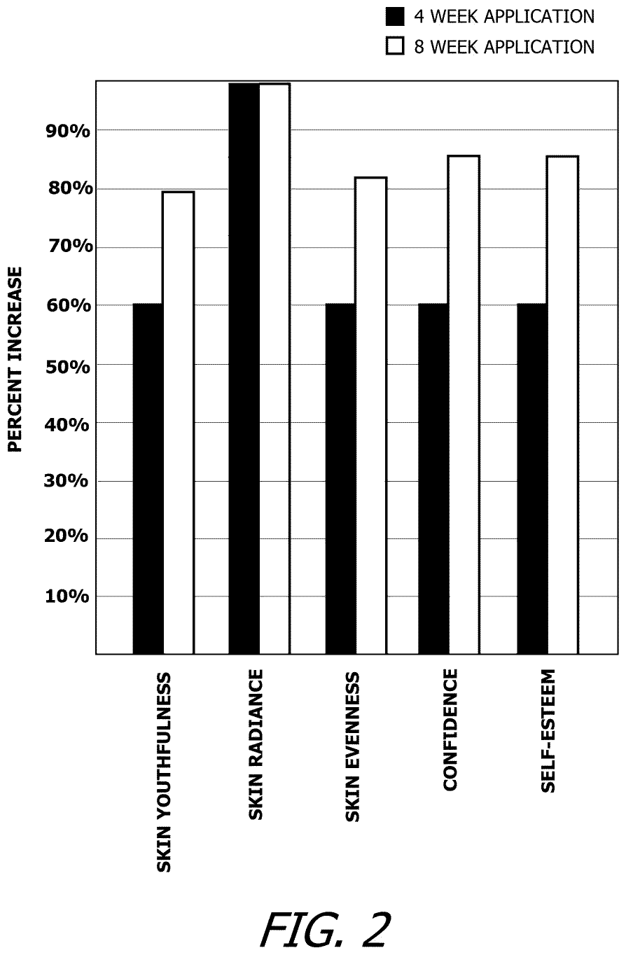

The topical formulation 12 both improves the condition and appearance of the skin and provides a hormonal response. The improvement in appearance and the hormonal response work in unison to improve the self-confidence and self-esteem of the user. Referring to FIG. 2 in conjunction with FIG. 1, it can be seen that users who applied the topical formulation 12 to their skin for 4-8 weeks reported more youthful and radiant skin at least sixty percent of the time. Likewise, the same users reported a corresponding increase in confidence and self-esteem.

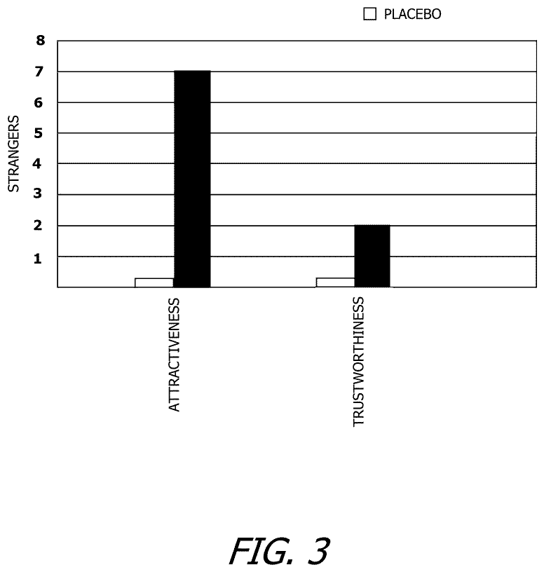

As has been explained, the first set 14 of active ingredients create a reaction in the body that induces the production of hormones and/or pheromones. Hormones, such as oxytocin and its kind promote improved social behavior and enhances positive social perceptions of others. Referring to FIG. 3, it can be seen that people under the influence of a hormone, such as oxytocin, consider unfamiliar faces with higher ratings of “trustworthiness” and “attractiveness” as compared to control groups who were not. Oxytocin also masks negative personality perceptions that masculinized faces tend to elicit.

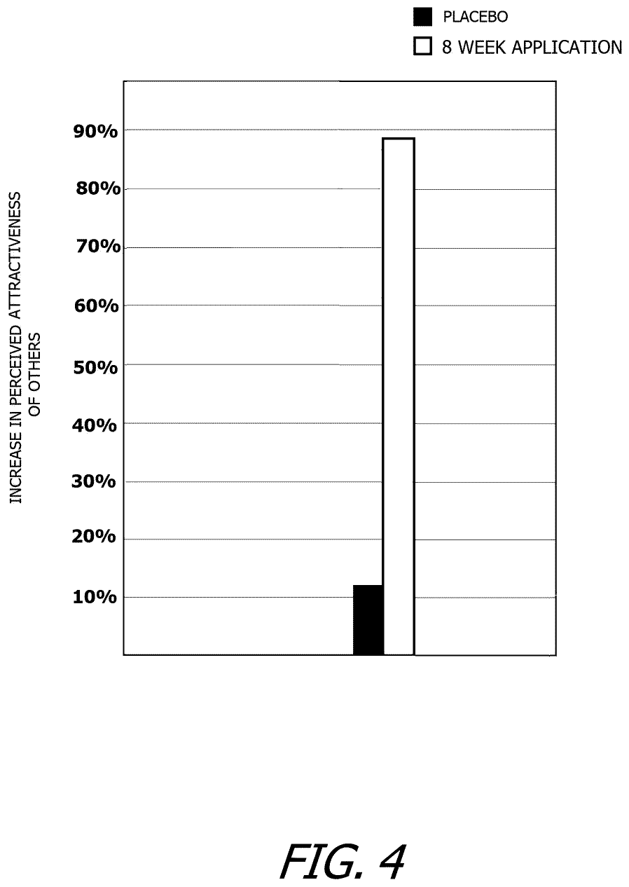

Referring to FIG. 4 it can be seen that when the topical formulation 12 is applied for four to eight weeks, users were 87.5% more likely to find random individuals of the opposite sex attractive, whereas the placebo group only found 12.5% of random individuals of the opposite sex more attractive.

The improved aesthetic appearance and hormonal response ultimately results in improved self-esteem and an enhanced quality of life. Moreover, enhancing aesthetic appearance correlates with improved first impressions and perceived attractiveness and success.

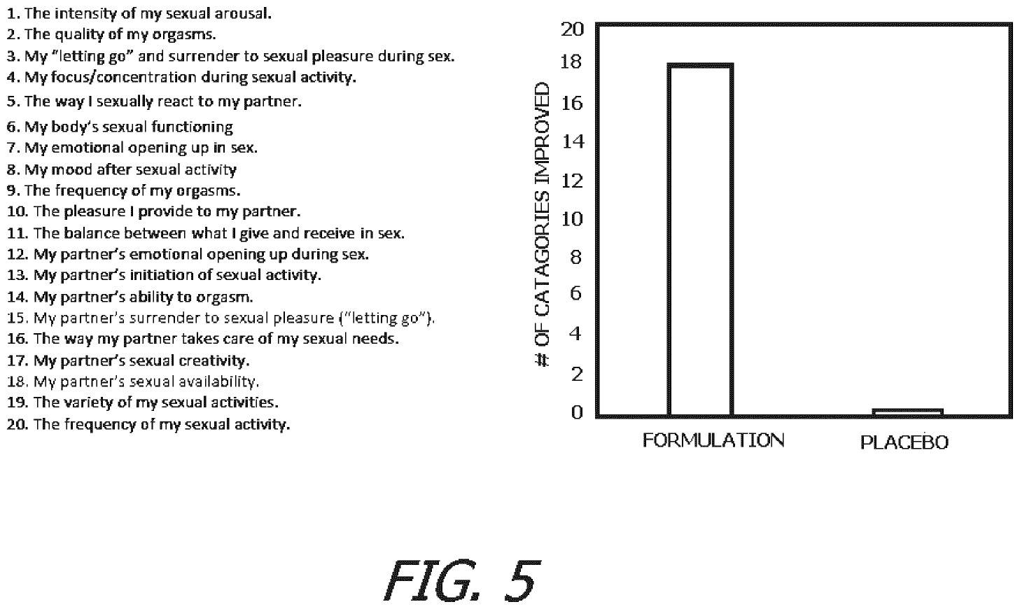

Referring to FIG. 5, a user survey 20 and a results graph 22 are shown. The user survey contains twenty categories relating to the sextual wellbeing of a user and the user; s partner. The twenty categories include sexual arousal, quality/quantity of orgasms, concentrations during sex, and the like. As can be seen from the graph 22, users of the topical formulation indicate improvements in eighteen out of the twenty categories. This is compared to a placebo, where only one category from the twenty showed improvement.

In addition to the hormonal/pheromonal response of the topical formulation, tactile stimulation has also been found to elevate plasma oxytocin levels, potentially enhancing fidelity in romantic partnerships. Additionally, since the topical formulation 12 induces a hormonal/pheromonal response, the sexual partners of formulation users also reported higher sexual satisfaction. This is due to the third aromatic pathway approach which is enhanced by the carob fruit extract. Accordingly, the combination of jasmine and carob derivatives, working through oxytocin and pheromonal pathways, found within the formulation 12, work synergistically enhance self-esteem, self-confidence, and sexual health. Simultaneously, the topical formulation 12 improves the condition and appearance of the skin, therein adding to self-esteem, self-confidence, and activeness to the opposite sex.

It will be understood that the embodiment of the present invention that is illustrated and described is merely exemplary and that a person skilled in the art can make many variations to that embodiment. For instance, many of the preferred ingredients listed in the topical formulation have equivalents that can be substituted in their place. Likewise, the concentrations of the various ingredients can be varied to adjust the viscosity and shelf-life of the formulations. All such embodiments are intended to be included within the scope of the present invention as defined by the appended claims.

What is claimed is:

1. A method of creating a hormonal response in the body using botanicals, said method comprising:
providing a topical formulation that contains jasmine flower extract, carob fruit extract and phospholipids, where said phospholipids enhance the absorption of said jasmine flower extract and said carob fruit extract into skin; and
applying said topical formulation onto the skin, wherein said jasmine flower extract and said carob fruit extract are in concentrations sufficient to induce a hormonal response that results in the release of oxytocin into the body.

2. The method according to claim 1, further including mixing peptides into said topical formulation to increase the ability of the skin to absorb said jasmine flower extract and said carob fruit extract.

3. The method according to claim 2, wherein said peptides include Palmitoyl Tripeptide-5.

4. The method according to claim 1, further including mixing at least one humectant into said topical formulation to moisturize the skin, wherein said humectant is selected from a group comprising propanediol and caprylyl glycol.

5. The method according to claim 1, further including mixing at least one emollient into said topical formulation to soften the skin.

6. The method according to claim 5, wherein said at least one emollient includes triheptanoin.

7. The method according to claim 1, further including mixing at least one soothing agent into said topical formulation to reduce inflammation of the skin, wherein said at least one soothing agent includes beta-glucan.

8. The method according to claim 1, further including mixing at least one surfactant into said topical formulation, wherein said at least one surfactant includes decyl glucoside.

9. The method according to claim 1, wherein said topical formulation is an aqueous solution that primarily contains water and glycerin.

10. A method of producing a topical formulation that induces a hormonal response in the body when applied to the skin, said method comprising:
mixing jasmine flower extract and carob fruit extract into an aqueous solution; and
adding phospholipids and peptides to said aqueous solution to enhance the ability of said topical formulation to be absorbed into the skin.

11. The method according to claim 10, wherein said peptides include Palmitoyl Tripeptide-5.

12. The method according to claim 10, further including mixing at least one humectant into said aqueous solution as a skin moisturizer, wherein said humectant is selected from a group comprising propanediol and caprylyl glycol.

13. The method according to claim 10, further including mixing at least one emollient into said aqueous solution.

14. The method according to claim 13, wherein said at least one emollient includes triheptanoin.

15. The method according to claim 10, further including mixing at least one anti-inflammatory agent into said topical solution to reduce inflammation of the skin, wherein said at least one anti-inflammatory agent includes beta-glucan.

16. The method according to claim 10, further including mixing at least one surfactant into said aqueous solution, wherein said at least one surfactant includes decyl glucoside.

17. The method according to claim 10, wherein said topical formulation primarily contains water and glycerin.

18. The method according to claim 10, further including mixing at least one chelating agent into said aqueous solution, wherein said at least one chelating agent includes trisodium ethylenediamine disuccinate.
